(19) United States
(12) Patent Application Publication
(10) Pub. No.: US 2022/0128566 A1
(43) Pub. Date: Apr. 28, 2022

(54) CALIBRATION OF SINGLE-MOLECULE DETECTION SYSTEM

(71) Applicant: Quantum-Si Incorporated, Guilford, CT (US)

(72) Inventors: Thomas Christian, Killingworth, CT (US); Kyle Preston, Guilford, CT (US); Brian Reed, Madison, CT (US); Shannon Stewman, Guilford, CT (US)

(21) Appl. No.: 17/511,258

(22) Filed: Oct. 26, 2021

Related U.S. Application Data
(60) Provisional application No. 63/152,005, filed on Feb. 22, 2021.
(60) Provisional application No. 63/106,313, filed on Oct. 27, 2020.

(51) Int. Cl. G01N 33/58; G01N 21/64

(52) U.S. Cl. CPC G01N 33/582; G01N 2021/6439; G01N 21/6428

(57) ABSTRACT

Aspects of the present disclosure relate to techniques for calibrating an integrated device. According to some embodiments, there is provided a method for calibrating an integrated device, the method comprising: exciting, with light from at least one excitation source, a reference dye molecule; obtaining a signal emitted by the reference dye molecule, the signal containing information representative of at least one characteristic of the reference dye molecule; and adjusting one or more subsequent measurements obtained from a sample based on the information obtained from the signal emitted by the reference dye molecule.

### CROSS-REFERENCE TO RELATED APPLICATIONS

This application claims the benefit under 35 U.S.C. § 119(e) of U.S. Provisional Patent Application Ser. No. 63/106,313 titled “CALIBRATION OF SINGLE-MOLECULE DETECTION SYSTEM” filed on Oct. 27, 2020 under Attorney Docket No. R0708.70113US00, and U.S. Provisional Application Ser. No. 63/152,005 titled “CALIBRATION OF SINGLE-MOLECULE DETECTION SYSTEM” filed on Feb. 22, 2021 under Attorney Docket No. R0708.70113US01, each of which is hereby incorporated by reference in their entireties herein.

### FIELD OF THE DISCLOSURE

The present disclosure relates to integrated devices and related instruments that can perform massively-parallel analyses of samples by providing short optical pulses to tens of thousands of reaction chambers or more simultaneously and receiving fluorescent signals from the reaction chambers for sample analyses. The instruments may be useful for point-of-care genetic sequencing and for personalized medicine.

### BACKGROUND

Photodetectors are used to detect light in a variety of applications. Integrated photodetectors have been developed that produce an electrical signal indicative of the intensity of incident light. Integrated photodetectors for imaging applications include an array of pixels to detect the intensity of light received from across a scene. Examples of integrated photodetectors include charge coupled devices (CCDs) and Complementary Metal Oxide Semiconductor (CMOS) image sensors.

Instruments that are capable of massively-parallel analyses of biological or chemical samples are typically limited to laboratory settings because of several factors that can include their large size, lack of portability, requirement of a skilled technician to operate the instrument, power need, need for a controlled operating environment, and cost. When a sample is to be analyzed using such equipment, a common paradigm is to extract a sample at a point of care or in the field, send the sample to the lab and wait for results of the analysis. The wait time for results can range from hours to days.

### SUMMARY OF THE DISCLOSURE

Some aspects of the present disclosure relate to a method for calibrating an integrated device, the method comprising: exciting, with light from at least one excitation source, a reference dye molecule; obtaining a signal emitted by the reference dye molecule, the signal containing information representative of at least one characteristic of the reference dye molecule; and adjusting one or more subsequent measurements obtained from a sample based on the information obtained from the signal emitted by the reference dye molecule to obtain one or more adjusted measurements.

Some embodiments provide for an integrated device comprising: at least one chamber for receiving a reference dye molecule; at least one photodetection region for receiving a signal emitted by the reference dye molecule when excited by light from at least one excitation source, the signal containing information representative of at least one characteristic of the reference dye molecule; and at least one controller configured to obtain one or more adjusted measurements by controlling adjusting of one or more subsequent measurements obtained from a sample disposed in the at least one chamber based on the information obtained from the signal emitted by the reference dye molecule.

A method of manufacturing an integrated device, comprising: providing a chamber on a substrate of the integrated device, the chamber being configured for receiving a reference dye molecule and positioned on the substrate such that the reference dye molecule receives light from at least one light source; providing a photodetection region positioned adjacent to the chamber such that the photodetection region receives a signal emitted by the reference dye molecule when the light from the at least one light source is delivered to the reference dye molecule; and coupling at least one controller to the photodetection region so that the at least one controller receives the signal emitted by the reference dye molecule, the at least one controller being configured to obtain information representative of at least one characteristic of the reference dye molecule from the signal, wherein the at least one controller is further configured to control adjusting one or more subsequent measurements obtained from a sample based on the information obtained from the signal emitted by the reference dye molecule to obtain one or more adjusted measurements.

### BRIEF DESCRIPTION OF DRAWINGS

The features and advantages of the present disclosure will become more apparent from the detailed description set forth below when taken in conjunction with the drawings. When describing embodiments in reference to the drawings, directional references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe a preferred or only orientation of features of an embodied device. A device may be embodied using other orientations.

FIG. 1-1A is a block diagram of an integrated device and an instrument, according to some embodiments.

FIG. 1-1B is a schematic of an integrated device, according to some embodiments.

FIG. 1-1C is a schematic of a pixel of an integrated device, according to some embodiments.

FIG. 1-1D is a circuit diagram of the pixel of FIG. 1-1C, according to some embodiments.

FIG. 1-1E is a top view of the pixel of FIG. 1-1C, according to some embodiments.

FIG. 1-1F is a plan view of the pixel of FIGS. 1-1C and 1-1D, according to some embodiments.

FIG. 1-1G is a schematic of an alternative pixel of an integrated device, according to some embodiments.

FIG. 1-1H is a circuit diagram of the pixel of FIG. 1-1G, according to some embodiments.

FIG. 1-1I is a plan view of the pixel of FIGS. 1-1C and 1-1D, according to some embodiments.

FIG. 2 illustrates an example process for calibrating an integrated device using emission information, according to some embodiments.

FIG. 3-1 illustrates a dye-labeled sample attached to a surface, according to some embodiments.

FIG. 3-2 illustrates a dye-labeled sample attached in a reaction chamber, according to some embodiments.

FIG. 4-1 illustrates a sequencing reaction in a reaction chamber, according to some embodiments.

FIG. 4-2 illustrates an example graph illustrating measured intensity and lifetime of emissions from sample molecules, according to some embodiments.

FIG. 5 illustrates example graphs illustrating measured intensity and fluorescence lifetime of emissions from sample molecules in different reaction chambers, according to some embodiments.

### DETAILED DESCRIPTION

### I. Introduction

Aspects of the present disclosure relate to techniques for calibrating an integrated device. According to some embodiments, the calibration techniques use emission information (e.g., signal intensity, fluorescence lifetime, fluorescence wavelength, pulse duration, interpulse duration, etc.) obtained by the integrated device from a reference dye molecule to adjust data used for sample identification (e.g., in sequencing applications).

The inventors have appreciated that, in instruments capable of massively-parallel analysis of biological or chemical samples by fluorescent emissions, it is desirable to increase the number of different dye-labeled affinity reagents that can be distinguished from each other. For example, in protein sequencing applications, it is desirable to distinguish between twenty amino acid residues in a multi-dimensional signal analysis by using emission characteristics, such as signal intensity, wavelength, and/or fluorescence lifetime, as well as kinetic characteristics of detected pulses such as pulse duration and/or interpulse duration. As the number of affinity reagents to be distinguished increases, the accuracy of distinguishing between them may suffer. This may be particularly true in a massively-parallel array where factors such as excitation intensity, collection efficiency, and photodetector properties may vary between different reaction chambers of an integrated device.

In addition, in some embodiments, it may be desirable to increase the number of samples which may be analyzed in parallel by multiplexing more than one sample with each photodetector of the integrated device. As the number of multiplexed samples increase, the accuracy of distinguishing between them may suffer.

Thus, the inventors have developed techniques for improving the accuracy of distinguishing between different samples, including different dye-labeled affinity reagents and/or different samples multiplexed on the same photodetector. According to some aspects, the techniques described herein perform a calibration process that extracts emission information from a reference dye loaded into a reaction chamber prior to performing sampling during a sequencing process. The emission information obtained from the reference dye may be used to localize clusters of emission information obtained from a molecule of interest during sequencing to account for differences among pixels of the integrated device that cause variations among signals emitted by samples located in the pixels.

According to some embodiments there is provided a method for calibrating an integrated device, the method comprising: exciting, with light from at least one excitation source, a reference dye molecule; obtaining a signal emitted by the reference dye molecule, the signal containing information representative of at least one characteristic of the reference dye molecule; and adjusting one or more subsequent measurements obtained from a sample based on the information obtained from the signal emitted by the reference dye molecule to obtain one or more adjusted measurements. The method may further comprise identifying the sample based at least in part on the one or more adjusted measurements.

In some embodiments, the at least one characteristic of the reference dye molecule comprises a signal intensity, a fluorescence wavelength, a fluorescence lifetime, a pulse duration, and/or an interpulse duration of the reference dye molecule.

In some embodiments, the reference dye molecule and the sample are disposed in a chamber of the integrated device.

In some embodiments, the one or more adjusted measurements are representative of a signal intensity, a fluorescence wavelength, a fluorescence lifetime, a pulse duration and/or an interpulse duration of the sample.

In some embodiments, the sample comprises a polypeptide. In some embodiments, identifying the sample comprises identifying one or more amino acids of the sample based at least in part on the one or more adjusted measurements, and identifying the polypeptide based at least in part on the one or more identified amino acids. In some embodiments, the one or more amino acids comprise at least 5 amino acids. In some embodiments, the one or more amino acids comprise no more than 50 amino acids. In some embodiments, the one or more amino acids comprise no more than a portion of the polypeptide. In some embodiments, wherein the polypeptide comprises a protein.

In some embodiments, the method further comprises labeling a first of the one or more amino acids with a first fluorescent label; exciting, with light from the at least one excitation source, the first fluorescent label; obtaining a signal emitted from the first fluorescent label containing information representative of a measure of at least one characteristic of the first fluorescent label; adjusting the measure of the at least one characteristic of the first fluorescent label based on the information obtained from the signal emitted by the reference dye molecule; and cleaving the first of the one or more amino acids from the sample. In some embodiments, the method further comprises, subsequent to cleaving the first of the one or more amino acids from the sample, obtaining a signal emitted from a second fluorescent label labeling a second of the one or more amino acids representative of a measure of at least one characteristic of the second fluorescent label; and adjusting the measure of the at least one characteristic of the second fluorescent label based on the information obtained from the signal emitted by the reference dye molecule.

In some embodiments, identifying the one or more amino acids of the sample comprises determining a type of the one or more amino acids. In some embodiments, identifying the one or more amino acids of the sample comprise determining an identity of the one or more amino acids. In some embodiments, identifying the one or more amino acids comprises determining an order of respective ones of the one or more amino acids relative to others of the one or more amino acids. In some embodiments, the sample comprises a fragment of the polypeptide. In some embodiments, identifying the one or more amino acids comprises comparing the one or more adjusted measurements to known information.

In some embodiments, the sample comprises a deoxyribonucleic acid (DNA) strand. In some embodiments, identifying the sample comprises identifying one or more nucleotides of the DNA strand based at least in part on the one or more adjusted measurements.

In some embodiments, the sample comprises a ribonucleic acid (RNA) strand. In some embodiments, identifying the sample comprises identifying one or more nucleotides of the RNA strand based at least in part on the one or more adjusted measurements.

In some embodiments, the one or more subsequent measurements comprise at least two subsequent measurements.

In some embodiments, adjusting the one or more subsequent measurements comprises determining an offset to be added to the one or more subsequent measurements.

In some embodiments, the reference dye molecule comprises a fluorescent molecule immobilized in the chamber of the integrated device.

In some embodiments, the integrated device comprises a plurality of chambers; the reference dye molecule comprises a plurality of reference dye molecules disposed in respective ones of the plurality of chambers; and obtaining a signal emitted by the reference dye molecule comprises obtaining a plurality of signals emitted by the plurality of reference dye molecules; the sample comprises a plurality of samples; and adjusting the one or more subsequent measurements from the sample comprises adjusting one or more subsequent measurements obtained respectively from each of the plurality of samples based on information obtained from the plurality of signals emitted by the plurality of reference dye molecules. In some embodiments, adjusting the one or more subsequent measurements comprises determining respective offsets for each of the one or more subsequent measurements based on the respective ones of the plurality of signals emitted by the plurality of reference dye molecules. In some embodiments, the plurality of reference dye molecules comprises a same molecule.

In some embodiments, obtaining one or more subsequent measurements obtained from the sample comprises: exciting, with light from the at least one excitation source, one or more fluorescent labels attached to the sample; and collecting one or more signals emitted from the one or more fluorescent labels attached to the sample, the one or more signals comprising the one or more subsequent measurements.

In some embodiments, the method further comprises loading the reference dye molecule into a chamber of the integrated device prior to exciting the reference dye molecule. In some embodiments, the sample is labeled with the reference dye molecule prior to exciting the reference dye molecule. In some embodiments, the method further comprises loading the sample into the chamber of the integrated device subsequent to loading the reference dye molecule into the chamber. In some embodiments, the method further comprises loading the sample into the chamber of the integrated device, wherein the loading of the reference dye molecule and the loading of the sample are performed in parallel. In some embodiments, loading the reference dye molecule into the chamber of the integrated device comprises immobilizing the reference dye molecule to a surface of the chamber. In some embodiments, the reference dye molecule is a freely diffusing dye molecule.

Some embodiments provide for an integrated device comprising: at least one chamber for receiving a reference dye molecule; at least one photodetection region for receiving a signal emitted by the reference dye molecule when excited by light from at least one excitation source, the signal containing information representative of at least one characteristic of the reference dye molecule; and at least one controller configured to obtain one or more adjusted measurements by controlling adjusting of one or more subsequent measurements obtained from a sample disposed in the at least one chamber based on the information obtained from the signal emitted by the reference dye molecule. In some embodiments, the at least one controller is further configured to identify the sample based at least in part on the one or more adjusted measurements.

In some embodiments, the at least one characteristic of the reference dye molecule comprises a signal intensity, a fluorescence wavelength, a fluorescence lifetime, a pulse duration, and/or an interpulse duration of the reference dye molecule.

In some embodiments, the one or more adjusted measurements are representative of a signal intensity, a fluorescence wavelength, a fluorescence lifetime, a pulse duration, and/or an interpulse duration of the sample.

In some embodiments, the sample comprises a polypeptide. In some embodiments, the at least one controller is configured to identify the sample at least in part by identifying one or more amino acids of the sample based at least in part on the one or more adjusted measurements, and identify the polypeptide based at least in part on the one or more identified amino acids. In some embodiments, the one or more amino acids comprise at least 5 amino acids. In some embodiments, the one or more amino acids comprise no more than 50 amino acids. In some embodiments, the one or more amino acids comprise no more than a portion of the polypeptide. In some embodiments, the polypeptide comprises a protein.

In some embodiments, a first of the one or more amino acids is labeled with a first fluorescent label; and the controller is configured to control adjusting of a measure of at least one characteristic of the first fluorescent label obtained by the photodetection region when the first fluorescent label is excited with light from the at least one light source based on the information obtained from the signal emitted by the reference dye molecule.

In some embodiments, the at least one controller is configured to identify the one or more amino acids of the sample at least in part by determining a type of the one or more amino acids. In some embodiments, the at least one controller is configured to identify the one or more amino acids of the sample at least in part by determining an identity of the one or more amino acids. In some embodiments, the at least one controller is configured to identify the one or more amino acids at least in part by determining an order of respective ones of the one or more amino acids relative to others of the one or more amino acids. In some embodiments, the sample comprises a fragment of the polypeptide. In some embodiments, the at least one controller is configured to identify the one or more amino acids at least in part by comparing the one or more adjusted measurements to known information.

In some embodiments, the sample comprises a deoxyribonucleic acid (DNA) strand. In some embodiments, the at least one controller is configured to identify the sample at least in part by identifying one or more nucleotides of the DNA strand based at least in part on the one or more adjusted measurements.

In some embodiments, the sample comprises a ribonucleic acid (RNA) strand. In some embodiments, the at least one controller is configured to identify the sample at least in part by identifying one or more nucleotides of the RNA strand based at least in part on the one or more adjusted measurements.

In some embodiments, the one or more subsequent measurements comprise at least two subsequent measurements. In some embodiments, the at least one controller is configured to control the adjusting of the one or more subsequent measurements at least in part by determining an offset to be added to the one or more subsequent measurements.

In some embodiments, the reference dye molecule comprises a fluorescent molecule immobilized in the chamber of the integrated device.

In some embodiments, the at least one chamber comprises a plurality of chambers; the reference dye molecule comprises a plurality of reference dye molecules disposed in respective ones of the plurality of chambers; the at least one photodetection region comprises a plurality of photodetection regions configured to receive signals from the plurality of reference dye molecules; and the sample comprises a plurality of samples, wherein the at least one controller is configured to control the adjusting of the one or more subsequent measurements obtained respectively from each of the plurality of samples based on information obtained from the plurality of signals emitted by the plurality of reference dye molecules.

In some embodiments, the at least one controller is configured to control the adjusting of the one or more subsequent measurements at least in part by determining respective offsets for each of the one or more subsequent measurements based on the respective ones of the plurality of signals emitted by the plurality of reference dye molecules. In some embodiments, the plurality of reference dye molecules comprises a same molecule.

In some embodiments, the one or more subsequent measurements are obtained by collecting, with the at least one photodetection region, signals emitted from one or more fluorescent labels attached to the sample when the sample is excited with light from the at least one excitation source.

In some embodiments, the sample is labeled with the reference dye molecule.

In some embodiments, wherein the reference dye molecule is a freely diffusing dye molecule.

A method of manufacturing an integrated device, comprising: providing a chamber on a substrate of the integrated device, the chamber being configured for receiving a reference dye molecule and positioned on the substrate such that the reference dye molecule receives light from at least one light source; and providing a photodetection region positioned adjacent to the chamber such that the photodetection region receives a signal emitted by the reference dye molecule when the light from the at least one light source is delivered to the reference dye molecule.

In some embodiments, the method further comprises coupling at least one controller to the photodetection region so that the at least one controller receives the signal emitted by the reference dye molecule, the at least one controller being configured to obtain information representative of at least one characteristic of the reference dye molecule from the signal. In some embodiments, the at least one controller is further configured to control adjusting one or more subsequent measurements obtained from a sample based on the information obtained from the signal emitted by the reference dye molecule to obtain one or more adjusted measurements.

In some embodiments, the method further comprises loading the reference dye molecule into the chamber. In some embodiments, the method further comprises labeling a sample with the reference dye molecule. In some embodiments, the method further comprises loading a sample into the chamber subsequent to loading the reference dye molecule into the chamber. In some embodiments, the method further comprises loading a sample into the chamber, wherein the loading of the reference dye molecule and the loading of the sample are performed in parallel. In some embodiments, loading the reference dye molecule into the chamber comprises immobilizing the reference dye molecule to a surface of the chamber. In some embodiments, the reference dye molecule is a freely diffusing dye molecule.

### II. Integrated Device Overview

The techniques described herein may be used to calibrate an integrated device. The integrated device may facilitate providing excitation light from one or more excitation sources located separate from a pixel array of an integrated device to a reaction chamber containing a sample. The excitation light may be directed at least in part by elements of the integrated device towards one or more pixels to illuminate an illumination region within the reaction chamber. A sample disposed in the reaction chamber, or a reaction component attached to the sample (such as a fluorescent label, for example), may emit emission light when located within the illumination region of the reaction chamber and in response to being illuminated by the excitation light. In some embodiments, the one or more excitation sources are part of a system comprising the integrated device.

Emission light emitted from one or more reaction chambers (e.g., at least two reaction chambers, in some embodiments), may be detected by one or more photodetectors within a pixel of the integrated device. As described herein, the integrated device may be configured having multiple pixels (e.g., an array of pixels), and thus, may have multiple reaction chambers and corresponding photodetectors. Characteristics of the detected emission light may provide an indication for identifying the label associated with the emission light. Such characteristics may include any suitable type of characteristic, including an arrival time of photons detected by the photodetector, an amount of photons accumulated over time by a photodetector, and/or a distribution of photons across two or more photodetectors. In some embodiments, a photodetector may have a configuration that allows for detection of one or more characteristics associated with the emission light, such as timing characteristics (e.g., fluorescence lifetime, pulse duration, interpulse duration), wavelength, and/or intensity. As one example, one or more photodetectors may detect a distribution of photon arrival times after a pulse of excitation light propagates through the integrated device, and the distribution of arrival times may provide an indication of a timing characteristic of the emission light (e.g., a proxy for fluorescence lifetime, pulse duration, and/or interpulse duration). Such information may be used in techniques for detection and/or identification of molecules in a sample, for example, including those described in U.S. patent application Ser. No. 16/686,028 titled “METHODS AND COMPOSITIONS FOR PROTEIN SEQUENCING,” filed Nov. 15, 2019 under Attorney Docket No. R0708.70042US02, PCT Application No. PCT/US19/61831 titled “METHODS AND COMPOSITIONS FOR PROTEIN SEQUENCING,” filed Nov. 15, 2019 under Attorney Docket No. R0708.70042WO00, U.S. patent application Ser. No. 62/984,229 titled “INTEGRATED SENSOR FOR MULTI-DIMENSIONAL SIGNAL ANALYSIS,” filed Mar. 2, 2020 under Attorney Docket No. R0708.70090US00, U.S. patent application Ser. No. 15/600,979 titled “LABELED NUCLEOTIDE COMBINATIONS AND METHODS FOR NUCLEIC ACID SEQUENCING,” filed May 22, 2017 under Attorney Docket No. R0708.70018US02, and U.S. patent application Ser. No. 15/161,125 titled “METHODS FOR NUCLEIC ACID SEQUENCING,” filed May 20, 2016 under Attorney Docket No. R0708.70020US00, each of which are incorporated by reference in their entireties. In some embodiments, one or more photodetectors provide an indication of a probability of emission light emitted by the fluorescent labels (e.g., fluorescence intensity). In some embodiments, one or more photodetectors may be sized and arranged to capture a spatial distribution of the emission light (e.g., wavelength). Output signals from the one or more photodetectors may be used to distinguish a fluorescent label from among a plurality of labels, where the plurality of labels may be used to identify a sample or it structure, as described herein.

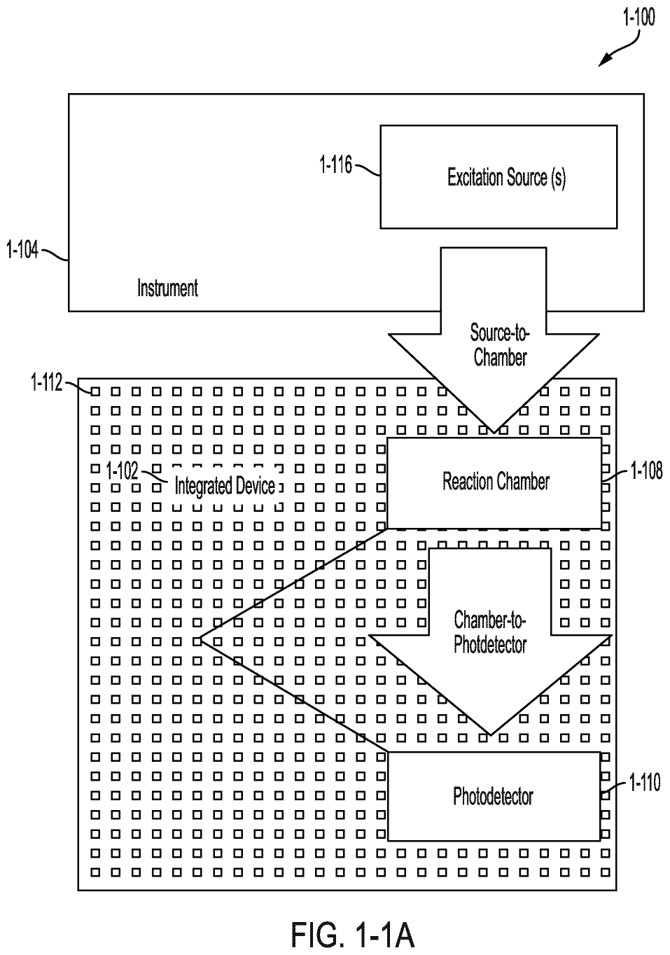

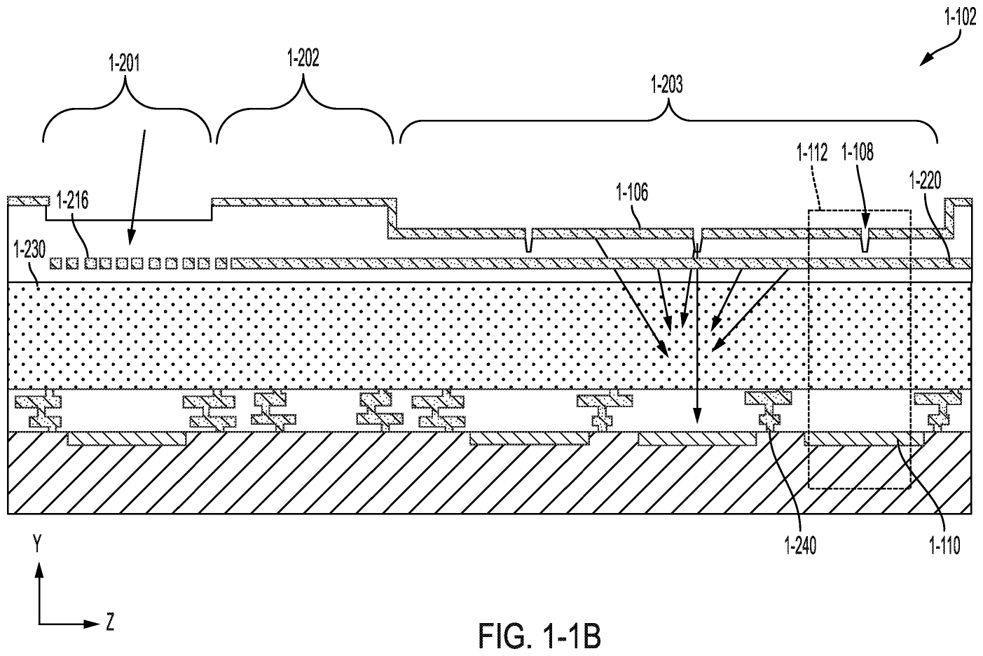

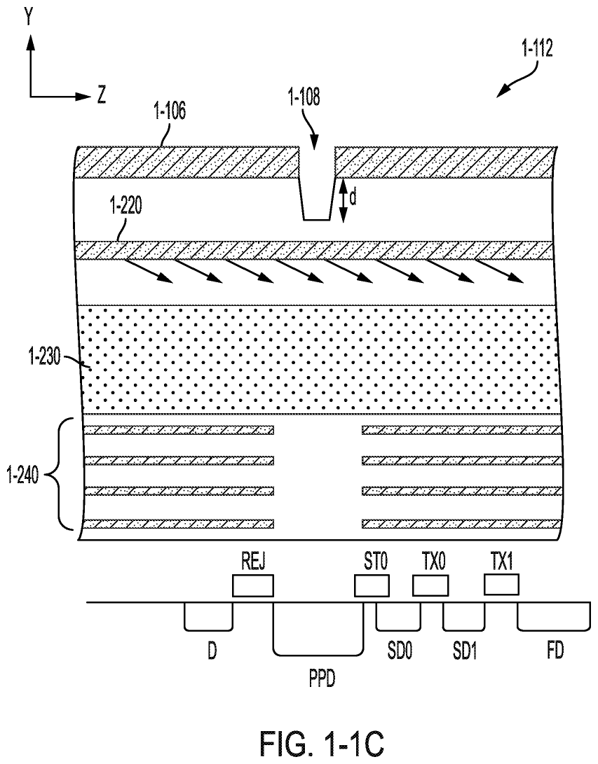

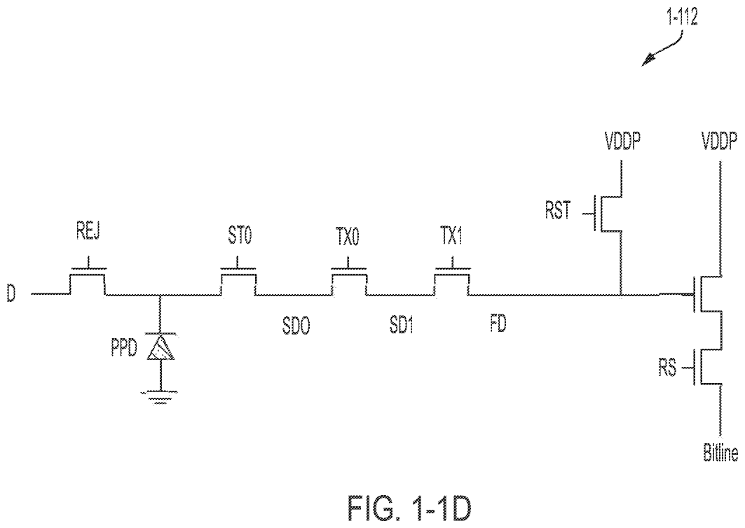

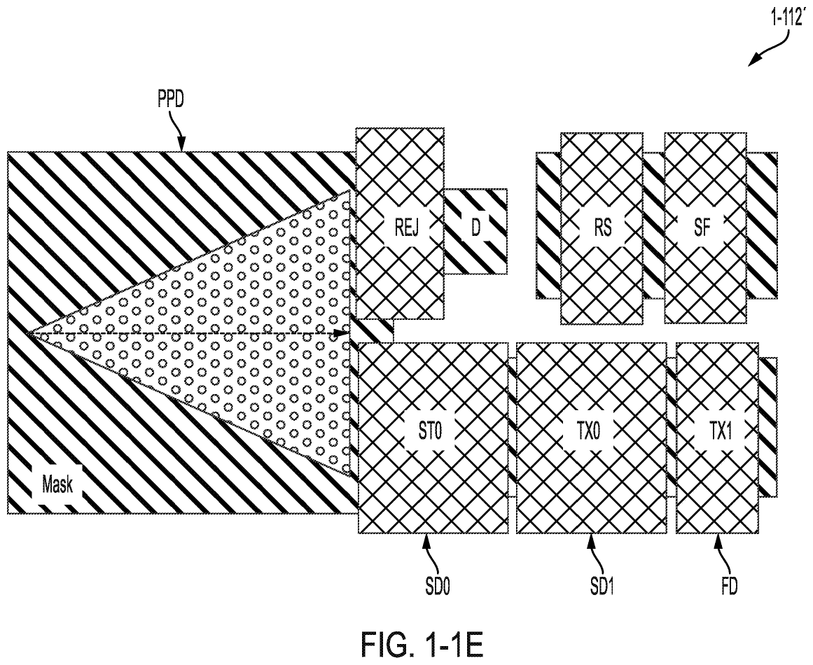

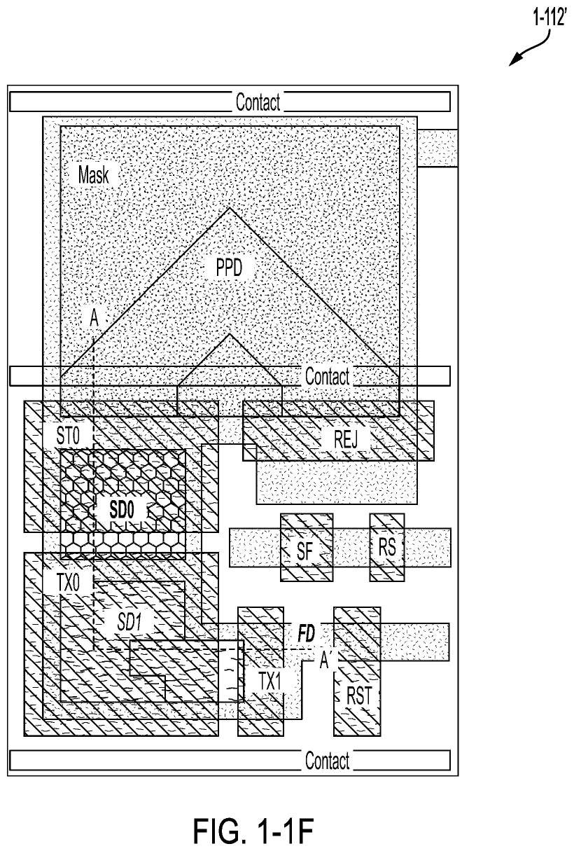

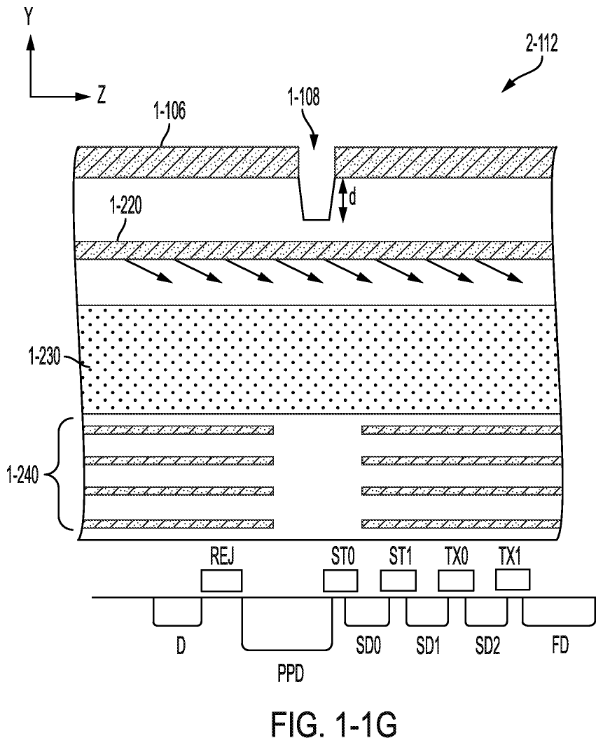

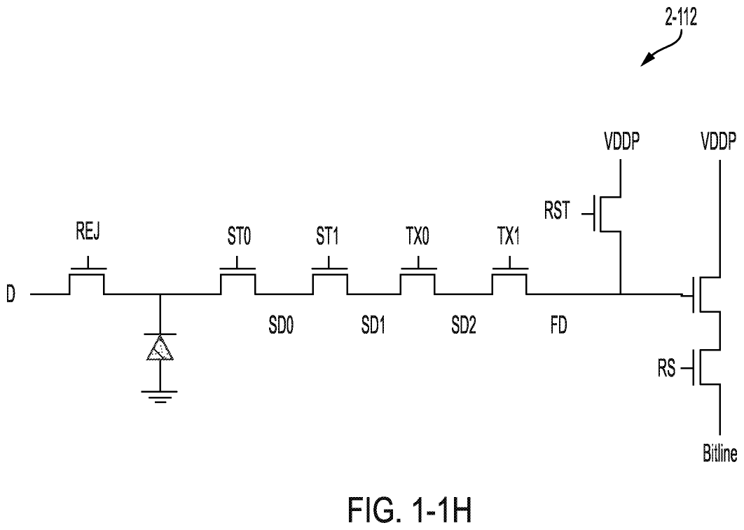

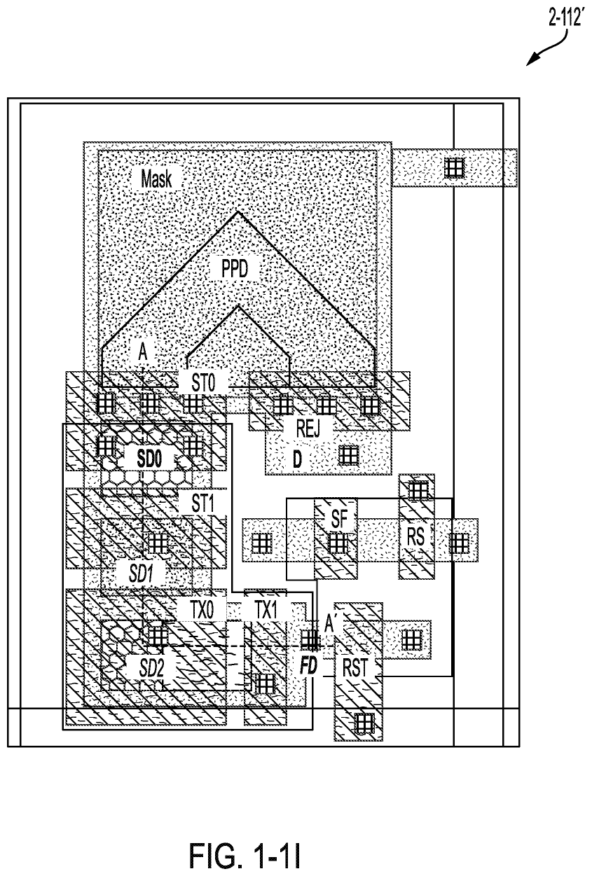

For example, a schematic overview of an exemplary system 1-100 is illustrated in FIG. 1-1A. The system comprises both an integrated device 1-102 that interfaces with an instrument 1-104. In some embodiments, instrument 1-104 may include one or more excitation sources 1-116 integrated as part of instrument 1-104. In some embodiments, an excitation source may be external to both instrument 1-104 and integrated device 1-102, and instrument 1-104 may be configured to receive excitation light from the excitation source and direct excitation light to the integrated device. The integrated device may interface with the instrument using any suitable socket for receiving the integrated device and holding it in precise optical alignment with the excitation source. The excitation source 1-116 may be configured to provide excitation light to the integrated device 1-102. As illustrated schematically in FIG. 1-1A, the integrated device 1-102 has a plurality of pixels 1-112, where at least a portion of pixels may perform independent analysis of a sample of interest. Such pixels 1-112 may be referred to as “passive source pixels” since a pixel receives excitation light from an excitation source 1-116 separate from the pixel, where excitation light from the source excites some or all of the pixels 1-112. Excitation source 1-116 may be any suitable light source. Examples of suitable excitation sources are described in U.S. patent application Ser. No. 14/821,688, filed Aug. 7, 2015, titled “INTEGRATED DEVICE FOR PROBING, DETECTING AND ANALYZING MOLECULES” under Attorney Docket Number R0708.70004US02, which is incorporated by reference in its entirety. In some embodiments, excitation source 1-116 includes multiple excitation sources that are combined to deliver excitation light to integrated device 1-102. The multiple excitation sources may be configured to produce multiple excitation energies or wavelengths.

Referring to FIG. 1-1B, a pixel 1-112 has a reaction chamber 1-108 configured to receive a at least one sample of interest and a photodetector 1-110 for detecting emission light emitted from the reaction chamber in response to illuminating the sample and at least a portion of the reaction chamber 1-108 with excitation light provided by the excitation source 1-116. In some embodiments, reaction chamber 1-108 may retain the sample in proximity to a surface of integrated device 1-102, which may ease delivery of excitation light to the sample and detection of emission light from the sample or a reaction component (e.g., a fluorescent label). As shown in the illustrated embodiment of FIG. 1-1B, the reaction chamber 1-108 and the photodetector 1-110 have a one-to-one correspondence. In some embodiments, as described herein, each pixel may comprise multiple reaction chambers per photodetector.

Optical elements for coupling excitation light from excitation light source 1-116 to integrated device 1-102 and guiding excitation light to the reaction chamber 1-108 may be located on one or both of the integrated device 1-102 and the instrument 1-104. Source-to-chamber optical elements may comprise one or more grating couplers located on integrated device 1-102 to couple excitation light to the integrated device and waveguides to deliver excitation light from instrument 1-104 to reaction chambers in pixels 1-112. One or more optical splitter elements may be positioned between a grating coupler and the waveguides. The optical splitter may couple excitation light from the grating coupler and deliver excitation light to at least one of the waveguides. In some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by reaction chambers of the integrated device.

Reaction chamber 1-108, a portion of the excitation source-to-chamber optics, and the reaction chamber-to-photodetector optics are located on integrated device 1-102. Excitation source 1-116 and a portion of the source-to-chamber components are located in instrument 1-104. In some embodiments, a single component may play a role in both coupling excitation light to reaction chamber 1-108 and delivering emission light from reaction chamber 1-108 to photodetector 1-110. Examples of suitable components, for coupling excitation light to a reaction chamber and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES” under Attorney Docket Number R0708.70004US02 and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES” under Attorney Docket Number R0708.70005US00, both of which are incorporated by reference in their entirety.

Pixel 1-112 is associated with its own individual reaction chamber 1-108 and at least one photodetector 1-110. The plurality of pixels of integrated device 1-102 may be arranged to have any suitable shape, size, and/or dimensions. Integrated device 1-102 may have any suitable number of pixels. The number of pixels in integrated device 1-102 may be in the range of approximately 100,000 pixels to 64,000,000 pixels or any value or range of values within that range. In some embodiments, the pixels may be arranged in an array of 1024 pixels by 2048 pixels. Integrated device 1-102 may interface with instrument 1-104 in any suitable manner. In some embodiments, instrument 1-104 may have an interface that detachably couples to integrated device 1-102 such that a user may attach integrated device 1-102 to instrument 1-104 for use of integrated device 1-102 to analyze at least one sample of interest in a suspension and remove integrated device 1-102 from instrument 1-104 to allow for another integrated device to be attached. The interface of instrument 1-104 may position integrated device 1-102 to couple with circuitry of instrument 1-104 to allow for readout signals from one or more photodetectors to be transmitted to instrument 1-104. Integrated device 1-102 and instrument 1-104 may include multi-channel, high-speed communication links for handling data associated with large pixel arrays (e.g., more than 10,000 pixels).

Instrument 1-104 may include a user interface for controlling operation of instrument 1-104 and/or integrated device 1-102. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, instrument 1-104 may include a computer interface configured to connect with a computing device. Computer interface may be a USB interface, a FireWire interface, or any other suitable computer interface. Computing device may be any general purpose computer, such as a laptop or desktop computer. In some embodiments, computing device may be a server (e.g., cloud-based server) accessible over a wireless network via a suitable computer interface. The computer interface may facilitate communication of information between instrument 1-104 and the computing device. Input information for controlling and/or configuring the instrument 1-104 may be provided to the computing device and transmitted to instrument 1-104 via the computer interface. Output information generated by instrument 5-104 may be received by the computing device via the computer interface. Output information may include feedback about performance of instrument 1-104, performance of integrated device 1-102, and/or data generated from the readout signals of photodetector 1-110.

In some embodiments, instrument 1-104 may include a processing device configured to analyze data received from one or more photodetectors of integrated device 1-102 and/or transmit control signals to excitation source(s) 1-116. In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof.) In some embodiments, the processing of data from one or more photodetectors may be performed by both a processing device of instrument 1-104 and an external computing device. In other embodiments, an external computing device may be omitted and processing of data from one or more photodetectors may be performed solely by a processing device of integrated device 1-102.

A cross-sectional schematic of integrated device 1-102 illustrating a row of pixels 1-112 is shown in FIG. 1-1B. Integrated device 1-102 may include coupling region 1-201, routing region 1-202, and pixel region 1-203. Pixel region 1-203 may include a plurality of pixels 1-112 having reaction chambers 1-108 positioned on a surface at a location separate from coupling region 1-201, which is where excitation light (shown as the dashed arrow) couples to integrated device 1-102. Reaction chambers 1-108 may be formed through metal layer(s) 1-106. One pixel 1-112, illustrated by the dotted rectangle, is a region of integrated device 1-102 that includes a reaction chamber 1-108 and a photodetection region having one or more photodetectors 1-110. In the illustrated embodiment, the pixel comprises a single reaction chamber 1-108. In some embodiments, each pixel may comprise two or more reaction chambers.

FIG. 1-1B illustrates the path of excitation (shown in dashed lines) by coupling a beam of excitation light to coupling region 1-201 and to reaction chambers 1-108. The row of reaction chambers 1-108 shown in FIG. 1-1B may be positioned to optically couple with waveguide 1-220. Excitation light may illuminate a sample located within a reaction chamber. The sample or reaction component (e.g., a fluorescent label) may reach an excited state in response to being illuminated by the excitation light. When a sample or reaction component is in an excited state, the sample or reaction component may emit emission light, which may be detected by one or more photodetectors associated with the reaction chamber. FIG. 1-1B schematically illustrates an optical axis of emission light (shown as the solid line) from a reaction chamber 1-108 to photodetector(s) 1-110 of pixel 1-112. The photodetector(s) 1-110 of pixel 1-112 may be configured and positioned to detect emission light from reaction chamber 1-108. Examples of suitable photodetectors are described in U.S. patent application 14/821,656, filed Aug. 7, 2015, titled “INTEGRATED DEVICE FOR TEMPORAL BINNING OF RECEIVED PHOTONS” under Attorney Docket Number R0708.70002US02, which is incorporated by reference in its entirety. For an individual pixel 1-112, a reaction chamber 1-108 and its respective photodetector(s) 1-110 may be aligned along a common axis (along the y-direction shown in FIG. 1-1A). In this manner, the photodetector(s) may overlap with the reaction chamber within a pixel 1-112.

The directionality of the emission light from a reaction chamber 1-108 may depend on the positioning of the sample in the reaction chamber 1-108 relative to metal layer(s) 1-106 because metal layer(s) 1-106 may act to reflect emission light. In this manner, a distance between metal layer(s) 1-106 and a fluorescent marker positioned in a reaction chamber 1-108 may impact the efficiency of photodetector(s) 1-110, that are in the same pixel as the reaction chamber, to detect the light emitted by the fluorescent marker. The distance between metal layer(s) 1-106 and the bottom surface of a reaction chamber 1-108, which is proximate to where a sample may be positioned during operation, may be in the range of 100 nm to 500 nm, or any value or range of values in that range. In some embodiments the distance between metal layer(s) 1-106 and the bottom surface of a reaction chamber 1-108 is approximately 300 nm.

The distance between the sample and the photodetector(s) may also impact efficiency in detecting emission light. By decreasing the distance light has to travel between the sample and the photodetector(s), detection efficiency of emission light may be improved. In addition, smaller distances between the sample and the photodetector(s) may allow for pixels that occupy a smaller area footprint of the integrated device, which can allow for a higher number of pixels to be included in the integrated device. The distance between the bottom surface of a reaction chamber 1-108 and photodetector(s) may be in the range of 1 μm to 15 μm, or any value or range of values in that range. It should be appreciated that, in some embodiments, emission light may be provided through other means than an excitation light source and a reaction chamber. Accordingly, some embodiments may not include reaction chamber 1-108.

Photonic structure(s) 1-230 may be positioned between reaction chambers 1-108 and photodetectors 1-110 and configured to reduce or prevent excitation light from reaching photodetectors 1-110, which may otherwise contribute to signal noise in detecting emission light. As shown in FIG. 1-1B, the one or more photonic structures 1-230 may be positioned between waveguide 1-220 and photodetectors 1-110. Photonic structure(s) 1-230 may include one or more optical rejection photonic structures including a spectral filter, a polarization filter, and a spatial filter. Photonic structure(s) 1-230 may be positioned to align with individual reaction chambers 1-108 and their respective photodetector(s) 1-110 along a common axis. Metal layers 1-240, which may act as a circuitry for integrated device 1-102, may also act as a spatial filter, or polarization filter, in accordance with some embodiments. In such embodiments, one or more metal layers 1-240 may be positioned to block some or all excitation light from reaching photodetector(s) 1-110.

Coupling region 1-201 may include one or more optical components configured to couple excitation light from an external excitation source, for example, excitation source(s) 1-116 illustrated in FIG. 1-1A. Coupling region 1-201 may include grating coupler 1-216 positioned to receive some or all of a beam of excitation light. Examples of suitable grating couplers are described in U.S. Pat. Application 62/435,693, filed Dec. 15, 2017, titled “OPTICAL COUPLER AND WAVEGUIDE SYSTEM” under Attorney Docket Number R0708.70021US00, and U.S. patent application Ser. No. 16/861,399, filed Apr. 29, 2020, titled “SLICED GRATING COUPLER WITH INCREASED BEAM ALIGNMENT SENSITIVITY” under Attorney Docket Number R0708.70071US01, each of which are hereby incorporated by reference herein in their entireties. Grating coupler 1-216 may couple excitation light to waveguide 1-220, which may be configured to propagate excitation light to the proximity of one or more reaction chambers 1-108. Alternatively, coupling region 1-201 may comprise other well-known structures for coupling light into a waveguide.

Components located off of the integrated device may be used to position and align the excitation source 1-116 to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM” under Attorney Docket Number R0708.70010US02, which is incorporated by reference in its entirety. Another example of a beam-steering module is described in U.S. Pat. Application 62/435,679, filed Dec. 14, 2017, titled “COMPACT BEAM SHAPING AND STEERING ASSEMBLY” under Attorney Docket Number R0708.70024US00, which is incorporated herein by reference.

A sample to be analyzed may be introduced into reaction chamber 1-108 of pixel 1-112. The sample may be a biological sample or any other suitable sample, such as a chemical sample. The sample may include multiple molecules and the reaction chamber may be configured to isolate a single molecule. In some instances, the dimensions of the reaction chamber may act to confine a single molecule within the reaction chamber, allowing measurements to be performed on the single molecule. Excitation light may be delivered into the reaction chamber 1-108, so as to excite the sample or at least one fluorescent marker attached to the sample or otherwise associated with the sample while it is within an illumination area within the reaction chamber 1-108.

In operation, parallel analyses of samples within the reaction chambers are carried out by exciting some or all of the samples within the wells using excitation light and detecting signals from sample emission with the photodetectors. Emission light from a sample may be detected by a corresponding photodetector and converted to at least one electrical signal. Information regarding various characteristics of the emission light (e.g., wavelength, fluorescence lifetime, intensity, pulse duration and/or any other suitable characteristic) may be collected and used for subsequent analysis, as described herein. The electrical signals may be transmitted along conducting lines (e.g., metal layers 1-240) in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

FIG. 1-1C illustrates a cross-sectional view of a pixel 1-112 of integrated device 1-102, according to some embodiments. FIG. 1-1D shows a circuit diagram of pixel 1-112. FIG. 1 sh-1E shows an exemplary array of pixels 1-112 and processing circuit 1-114, which may be included in integrated device 1-102, according to some embodiments.

In FIGS. 1-1C and 1-1D, pixel 1-112 includes a photodetection region, which may be a pinned photodiode (PPD), two charge storage regions, which may be storage diodes (SD0 and SD1), and a readout region, which may be a floating diffusion (FD) region. Also as shown, pixel 1-112 also includes drain region D and transfer gates ST0, TX0, TX1, and REJ.

In some embodiments, photodetection region PPD, charge storage regions SD0 and SD1 and readout region FD may be formed on an integrated circuit substrate by doping parts of the substrate. For example, the substrate may be lightly doped and photodetection region PPD, charge storage regions SD0 and SD1, and readout region FD may be more heavily doped. In this example, the substrate may be lightly p-type doped and photodetection region PPD, charge storage regions SD0 and SD1, and readout region FD may be n-type doped. Alternatively, the substrate may be lightly n-type doped and photodetection region PPD, charge storage regions SD0 and SD1, and readout region FD may be p-type doped, as embodiments described herein are not so limited.

In some embodiments photodetection region PPD may be configured to generate charge carriers (e.g., photo-electrons) when incident photons are received therein. In some embodiments, charge storage regions SD0 and SD1 may be electrically coupled to photodetection region PPD and/or to one another. For example, pixel 1-112 may include one or more transfer channels electrically coupling charge storage regions SD0 and SD1 to photodetection region PPD and/or to one another. In some embodiments, the transfer channels may be formed by doping portions of the integrated circuit substrate disposed between the regions. For example, the portions may be doped with a same conductivity type as the regions (e.g., an n-type doped channel disposed between an n-type doped PPD and SD0). Referring to FIG. 1-1D, for example, a channel of a transistor coupled between photodetection region PPD and charge storage region SD0 is a transfer channel electrically coupling photodetection region PPD to charge storage region SD0. Similarly, a channel of a transistor coupled between charge storage regions SD0 and SD1 is a transfer channel electrically coupling charge storage region SD0 to SD1, a channel of a transistor coupled between charge storage region SD1 and readout region FD is a transfer channel electrically coupling charge storage region SD1 to readout region FD. A channel of a transistor coupled between photodetection region PPD and drain region D is a transfer channel between photodetection region PPD and drain region D.

In some embodiments, transfer gates ST0, TX0, TX1, and REJ may be configured to control the transfer of charge carriers from photodetection region PPD to storage regions SD0 and SD1, between charge storage regions SD0 and SD1, and/or between charge storage regions SD0 and SD1 and readout region FD. For example, transfer gates ST0, TX0, TX1, and REJ may be electrically coupled to and configured to bias the transfer channels electrically coupling the regions of pixel 1-112 to transfer the charge carriers between the regions when appropriate control signals are applied to the transfer gates. The transfer gates may me conductively (e.g., physically) coupled to the transfer channels, and/or may be positioned close enough to the transfer channels and/or separated by a thin enough insulator to capacitively couple to the transfer channels, according to various embodiments. In some embodiments, transfer gates described herein may be formed using a conductive material such as metal. Alternatively or additionally, in some embodiments, transfer gates described herein may be formed using a semiconductor material such as polysilicon. In some embodiments, materials used to form transfer gates described herein may be at least partially opaque.

In some embodiments, when a control signal is received at a transfer gate, the transfer gate may electrically couple the control signal to the transfer channel and bias the transfer channel, thereby increasing the conductivity of the transfer channel. In some embodiments, the transfer channel may be doped with a same conductivity type but a lower dopant concentration than the regions of pixel 1-112 electrically coupled by the transfer channel, thereby generating an intrinsic electric potential barrier between the regions. The intrinsic electric potential barrier may exist between the regions even when no external electric field is applied to the transfer gate or transfer channel. For example, the dopant concentration of the transfer channel between photodetection region PPD and charge storage region SD0 may generate an intrinsic electric potential barrier between photodetection region PPD and charge storage region SD0. In some embodiments, a control signal may be applied to the transfer gate, the control signal being configured to lower the intrinsic electric potential barrier between the regions electrically coupled by the transfer channel, thereby increasing the conductivity of the transfer channel, and causing a transfer of charge carriers between the regions. For example, for an n-type doped transfer channel, the control signal may have a voltage that is greater than a voltage at one of the regions (e.g., at the source terminal of the transfer channel) by at least a threshold voltage of the transfer channel, the threshold voltage being dependent on the size of the transfer channel, a substrate voltage of the integrated device 1-102 proximate the transfer channel, and other such parameters. Similarly, for a p-type doped transfer channel, the control signal may have a voltage that is lower than the voltage at the one of the regions by at least the threshold voltage. In some embodiments, a control circuit of integrated device 1-102 may be configured to generate and provide such control signals to the transfer gates, as described further herein.

In FIG. 1-1C, pixel 1-112 is shown in a configuration configured to receive incident photons in a direction in which photodetection region PPD, charge storage regions SD0 and SD1, and readout region FD are spaced from transfer gates REJ, ST0, TX0, and TX1 (e.g., front-side illumination). It should be appreciated, however, that in some embodiments, photodetection region PPD may be configured to receive incident photons in a direction in which transfer gates REJ, ST0, TX0, and TX1 are spaced from photodetection region PPD, charge storage regions SD0 and SD1, and readout region FD (e.g., back-side illumination). In some embodiments, such a configuration may improve the electrical characteristics of the transfer gates because the optical characteristics of the transfer gates have a reduced impact on the incident photons.

In FIG. 1-1D, pixel 1-112 further includes a reset (RST) transfer gate coupled to readout region FD and configured for coupling to a high voltage VDDP, and a row select (RS) transfer gate coupled between readout region FD and a bitline. When the integrated device 1-102 is coupled to a power source (e.g., at least a DC power supply), transfer gate RST may be coupled to high voltage VDDP, which is supplied by the power source and/or regulated by a voltage regulator of integrated device 1-102.

In some embodiments, transfer gate RST may be configured to reset a voltage of readout region FD. For example, when a reset signal is applied to transfer gate RST, transfer gate RST may bias the transfer channel electrically coupling readout region FD to high voltage VDDP, thereby increasing the conductivity of the transfer channel and transferring charge carriers from readout region FD to high voltage VDDP. In some embodiments, reset transfer gate RST may be further configured to reset the voltage of charge storage region SD0 and/or SD1. For example, when a reset signal is applied to reset transfer gate RST and a control signal is applied to transfer gate TX1, transfer gate TX1 may transfer charge carriers in charge storage region SD1 to readout region FD and transfer gate RST may transfer the charge carriers to high voltage VDDP. Similarly, when a reset signal is applied to reset transfer gate RST and control signals are applied to transfer gates TX1 and TX0, transfer gate TX0 may transfer charge carriers in charge storage region SD0 to SD1, transfer gate TX1 may transfer the charge carriers in charge storage region SD1 to readout region FD, and transfer gate RST may transfer the charge carriers to high voltage VDDP. In some embodiments, integrated device 1-102 may be configured to reset readout region FD and charge storage regions SD0 and SD1 before collecting and reading out charge carriers. For example, integrated device 1-102 may be configured to reset readout region FD, then reset charge storage region SD1, and then reset charge storage region SD0, before collecting and reading out charge carriers.

In some embodiments, the bitline may be coupled to processing circuitry on the integrated device 1-102 and/or an external circuit configured to receive a voltage level indicative of charge carriers read out to readout region FD. In some embodiments, processing circuitry 1-114 may include an analog-to-digital converter (ADC). In some embodiments, integrated device 1-102 may be configured to reset the voltage of readout region FD of each pixel before reading out charge carriers. For example, integrated device 1-102 may be configured to reset the voltage of readout region FD, sample the voltage, transfer charge carriers into readout region FD, and sample the voltage again. In this example, the second sampled voltage may be indicative of a number of the charge carriers transferred into readout region FD when compared to the first sampled voltage. In some embodiments, integrated device 1-102 may be configured to read out charge carriers from each pixel 1-112 to the bitline sequentially, such as row by row and/or column by column. It should be appreciated that some arrays of pixels 1-112 may have multiple bitlines electrically coupled to different ones and/or groups of pixels 1-112. In some embodiments, pixels of multiple columns may be read out to respective processing circuitry at the same time. For example, a first pixel of each column (e.g., pixels (1,1) and (1,2) and so on) may be read out to the respective processing circuitry at the same time, and then a second pixel of each column (e.g., pixels (2,1) and (2,2,) and so on) may be read out to the respective processing circuitry at the same time. It should be appreciated that, in some embodiments, processing circuitry may be provided for each row of the array as an alternative or in addition to each column. In some embodiments, integrated device 1-102 may include multiple units of processing circuitry, such as each being electrically coupled to a bitline.

It should be appreciated that, in accordance with various embodiments, transfer gates described herein may include semiconductor material(s) and/or metal, and may include a gate of a field effect transistor (FET), a base of a bipolar junction transistor (BJT), and/or the like. It should also be appreciated that control signals described herein applied to the various transfer gates may vary in shape and/or voltage, such as depending on the electric potential of the semiconductor region and of the regions electrically coupled to the semiconductor region (e.g., neighboring regions).

In some embodiments, pixels described herein may include more than two charge storage regions. For example, pixel 2-112 described herein in connection with FIGS. 1-1G-1-1I includes three charge storage regions.

FIG. 1-1E is a plan view of alternative pixel 1-112′, according to some embodiments. In some embodiments, pixel 1-112′ may be configured in the manner described for pixel 1-112. In FIG. 1-1E, drain region D of pixel 1-112′ is positioned on a same side of photodetection region PPD as charge storage regions SD0 and SD1 and readout region FD. Also shown in FIG. 1-1E, photodetection region PPD may include a mask with a triangular opening, with a base of the triangular opening on a side of photodetection region proximate charge storage regions SD0 and SD1 and drain region D, and a corresponding apex of the triangular opening on a side of photodetection region PPD opposite drain region D and charge storage regions SD0 and SD1.

In some embodiments, photodetection region PPD may be configured to induce an intrinsic electric field in a direction from photodetection region PPD toward charge storage regions SD0 and SD1 and drain region D. For example, photodetection region PPD may be formed by doping a substrate of integrated device 1-102 through the opening, resulting in a higher dopant concentration in the region of the substrate exposed through the opening than in the region covered by the mask during doping. In this example, the larger quantity of dopants (e.g., n-type dopants) at the base end of the triangular opening may cause the electric potential at the base end of photodetection region PPD proximate drain region D and charge storage region SD0 to be lower than the electric potential at the apex end of photodetection region PPD on the opposite side of photodetection region PPD. The intrinsic electric field in photodetection region PPD may be present even in the absence of an external electric field being applied to pixel 1-112. The inventors recognized that the intrinsic electric field of photodetection region PPD increases the rate of charge transfer from photodetection region PPD to drain region D and/or storage regions SD0 and SD1, increasing the efficiency with which charge carriers are drained and/or collected during operation of pixel 1-112. In the example of FIG. 1-1E, the intrinsic electric field may be directed along the dotted arrow between drain region and charge storage region SD0. For example, the intrinsic electric field may cause charge carriers to flow along the dotted arrow, and an extrinsic electric field induced by a control signal being applied to transfer gate REJ or ST0 may cause the charge carriers to flow to drain region D or charge storage region SD0, respectively.

FIG. 1-1F is a top schematic view of the pixel 1-112′, according to some embodiments. As shown in FIG. 1-1F, contacts may be disposed over portions of pixel 1-112′. In some embodiments, the contacts may be configured to block incident photons from reaching portions of pixel 1-112′ other than photodetection region PPD and/or from reaching photodetection regions of neighboring pixels at oblique angles of incidence. For example, the contacts may be elongated in a direction parallel to the optical axis along which photodetection PPD is configured to receive incident photons. In some embodiments, the contacts may be formed using an opaque material such as tungsten. The inventors have recognized that contacts described herein prevent many or all incident photons from reaching charge storage regions SD0 and SD1 along optical paths other than the optical axis, thereby preventing the incident photons from generating noise charge carriers in charge storage regions SD0 and SD1.

In FIG. 1-1F, a pair of contacts is disposed on opposite sides of photodetection region PPD, with a first contact of the pair disposed closer to the apex of the triangular opening of the mask and a second contact of the pair disposed closer to the base of the triangular opening of the mask. The second contact may be configured to block incident photons from reaching charge storage regions SD0 and SD1. A third contact is disposed at an end of pixel 1-112 opposite the end at which photodetection region PPD is disposed. The first and third contacts are disposed between the pixel 1-112 and respective neighboring pixels, and the second contact is positioned between photodetection region PPD and transfer gates ST0 and REJ. It should be appreciated that, in some embodiments, the pair of contacts on opposite sides of photodetection region PPD may be replaced with at least one contact wall that at least partially surrounds photodetection region PPD, such as a single cylindrical contact wall.

FIG. 1-1G is a cross-sectional view of an alternative example pixel 2-112, which may be included in integrated device 1-102, according to some embodiments. In some embodiments, pixel 2-112 may be configured in the manner described for pixel 1-112 in connection with FIGS. 1-1A-1-1F. For example, as shown in FIG. 1-1G, region FD, and drain region D, and transfer gates, each of which may be configured in the manner described for pixel 1-112. Pixel 2-112 further includes charge storage region SD2 electrically coupled between charge storage region SD1 and readout region FD. For example, transfer channels may electrically couple charge storage region SD1 to charge storage region SD2 and charge storage region SD2 to readout region FD. Transfer gate ST1 may electrically couple charge storage region SD0 to charge storage region SD1. In FIG. 1-1G, transfer gate TX0 is configured to control a transfer of charge carriers from charge storage region SD1 to charge storage region SD2, and transfer gate TX1 is configured to control a transfer of charge carriers from charge storage region SD2 to readout region FD.

FIG. 1-1H is a circuit diagram of pixel 2-112, according to some embodiments. As shown in FIG. 1-1H, the transfer channel electrically coupling charge storage region SD1 to charge storage region SD2 is a channel of a transistor having transfer gate TX0 and the transfer channel electrically coupling charge storage region SD2 to readout region FD is a channel of a transistor having transfer gate TX1. The other transistors of pixel 2-112 shown in FIG. 1-1H, such as the transistor having reset gate RST and the transistor having row select transfer gate RS may be configured in the manner described for pixel 1-112 in connection with FIGS. 1-3A and 1-3B. For example, an array of pixels 2-112 may be arranged in a configuration with processing circuitry as described herein for pixel 1-112 in connection with FIGS. 1-3B and 1-3C.

FIG. 1-1I is a top view of pixel 2-112′, which may be included in integrated device 1-102, according to some embodiments. In some embodiments, pixel 2-112′ may be configured in the manner described herein for pixel 1-112′. For example, photodetection region PPD of pixel 2-112′ may be configured to induce an intrinsic electric field in the direction from photodetection region PPD toward charge storage region SD0 and drain region D.

### III. Integrated Device Calibration Techniques

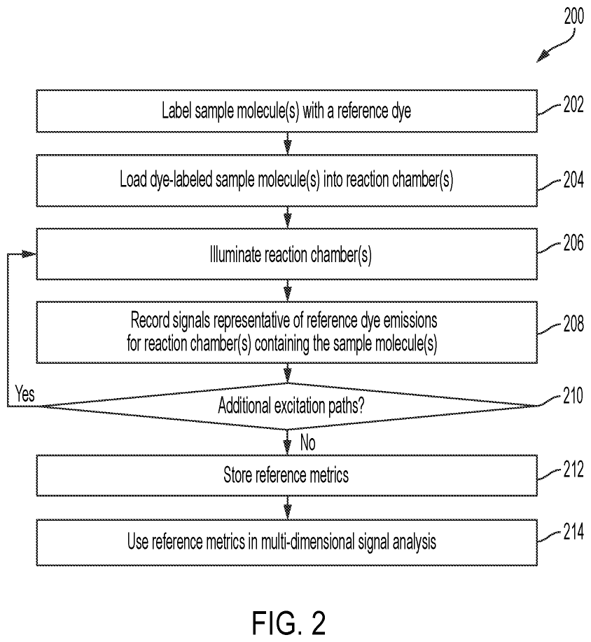

Aspects of the technology described herein relate to techniques for calibrating an integrated device using emission information obtained from a reference dye. FIG. 2 illustrates an example process 200 for calibrating an integrated device using emission information, according to some embodiments.

At act 202, a sample molecule may be labeled with a reference dye. For example, the reference dye may comprise a fluorescent molecule that emits emission light in response to excitation by light from at least one light source. The reference dye molecule may be of a type that binds to a particular sample molecule such that characteristics of the emission light emitted by the reference dye molecule may be used to identify the sample molecule to which the reference dye molecule is bound.

At act 204, the dye-labeled sample molecule is loaded into a reaction chamber. For example, in some embodiments, multiple reference dyes of the same type are loaded into a plurality of reaction chambers to evaluate differences in signals received from the plurality of reaction chambers. In some embodiments, the sample molecule may be labeled with the reference dye after the sample molecule is loaded into the reaction chamber, as aspects of the technology described herein are not limited in this respect.

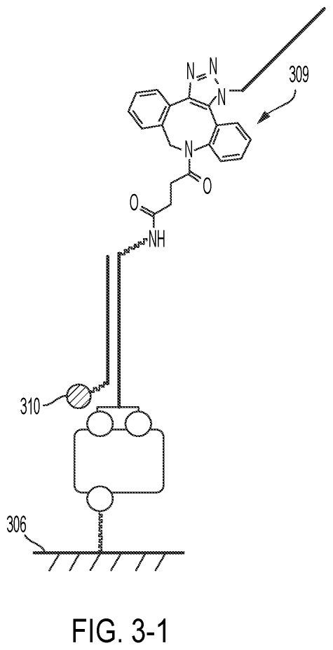

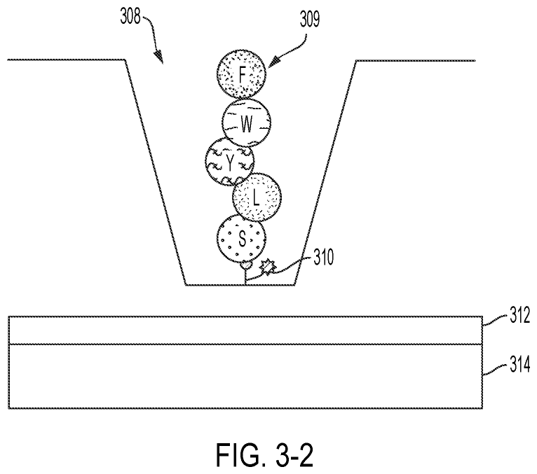

FIG. 3-1 illustrates a sample molecule 309 labeled with a reference dye molecule 310 attached to a surface 306, according to some embodiments. FIG. 3-2 illustrates a dye-labeled sample attached to a surface of a reaction chamber, according to some embodiments. In particular, in FIG. 3-2, a sample 309 comprising a chain of amino acids (phenylalanine (F), tryptophan (W), tyrosine (Y), leucine (L), and serine (S)) is loaded into the reaction chamber 308. The sample 309 is labeled with a reference dye molecule 310. A CMOS chip 314 comprising photonic components 312 for collecting signals emitted by the reference dye molecule 310 may be provided, as described herein with reference to the integrated device overview.

Act 204 may be performed while the reaction chamber(s) is not being illuminated with excitation light. It should be appreciated that, in some embodiments, multiple dye-labeled sample molecules may be loaded into a plurality of reaction chambers at act 204.

In some embodiments, the sample molecule with the reference dye may be loaded into a reaction chamber alone, and a molecule to be sequenced may be loaded into the reaction chamber thereafter. In some embodiments, the molecule with the reference dye may be loaded into the reaction chamber together with the molecule to be sequenced subsequent to performing the calibration process. In some embodiments, the reference dye may be attached to the molecule to be sequenced itself, and the reference dye may be cleaved prior to performing sequencing. In some embodiments, the reference dye may be a freely diffusing dye molecule which need not be attached or otherwise immobilized in the reaction chamber. In some embodiments, the reference dye may be loaded into a reaction chamber as part of the surface chemistry preparation of the chip. Thus, aspects of the technology are not limited to the particular configuration of the reference dye in the reaction chamber. According to some embodiments, act 202 may include techniques are described in U.S. Pat. Application No. 62/926,975, filed Oct. 28, 2019, titled “METHODS OF PREPARING SAMPLES FOR MULTIPLEX POLYPEPTIDE SEQUENCING” under Attorney Docket Number R0708.70077US00, which is incorporated by reference in its entirety.

At act 206, the reaction chamber(s) is illuminated with excitation light from a light source (e.g., a laser). In particular, at act 206 the reaction chamber(s) may be illuminated with excitation light (e.g., by unblocking the light source, recoupling the light source into the chip, and/or increasing the power delivered by the light source in a time that is fast relative to a characteristic bleaching time of the system) such that the reference dye molecule 310 emits a signal comprising photons. The reaction chamber may be illuminated for any suitable duration. In some embodiments, the excitation light may be delivered to the reaction chambers until the reference dye has been bleached.

At act 208, signals emitted by the reference dye are obtained by the photodetectors of the integrated device for the reaction chambers containing the sample molecule(s) illuminated at act 206. For example, in some embodiments, a reference signal intensity is obtained for each reaction chamber illuminated at act 206. The reference signal intensity may be extracted as an amplitude of the reference dye emission signal. The reference signal intensity may be a factor of excitation intensity at the reaction chamber and collection efficiency between the reaction chamber and the photodetector. In some embodiments, a reference fluorescence lifetime of the reference dye emission is measured by photodetectors of the integrated device. In some embodiments, a reference fluorescence wavelength of the reference dye emission is measured by photodetectors of the integrated device. In other embodiments, one or more other characteristics may additionally or alternatively be extracted from the reference dye emission signal, such as pulse duration and/or interpulse duration.

At act 210, it is determined whether there are additional excitation paths to excite, including one or more additional reaction chambers to illuminate. If, at act 210, it is determined that there are additional excitation paths to excite, the process 200 returns through the yes branch to act 206 to illuminate additional reaction chambers with the additional excitation paths. If, at act 210, it is determined that there are no additional excitation paths, the process 200 may proceed through the no branch to act 212.

At act 212, reference metrics may be stored. For example, the reference metrics may include information obtained at act 208. In some embodiments, the reference metrics may be stored locally within the integrated device. In some embodiments, the reference metrics may be stored in a memory remote from the integrated device.

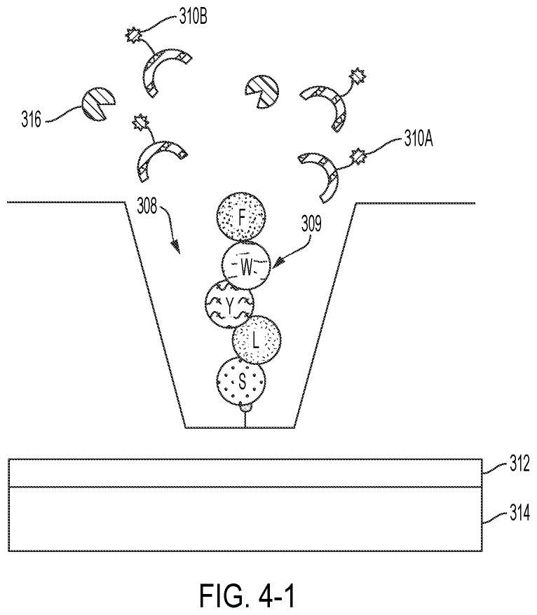

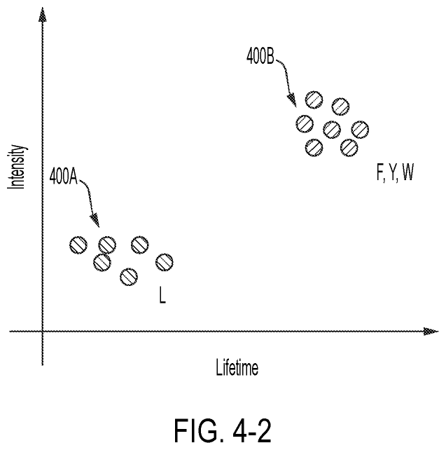

At act 214, the reference metrics may be used to assist in analyzing emissions information obtained from a sample during sequencing. For example, subsequent to performing the calibration process 200, the integrated device may be used to collect and analyze signals from molecules disposed in the reaction chamber(s). FIG. 4-1 illustrates a sequencing reaction in a reaction chamber which may be performed subsequent to the calibration process 200, according to some embodiments. The sequencing reaction shown in FIG. 4-1 may include attachment of reference dye molecules 310A, 310B to a sample 309 in a reaction chamber 308. As described herein, the reference dye molecules may be of a particular type, for example, binding to a particular type of sample molecule. For example, reference dye molecule 310A selectively binds only to a first type of amino acids (e.g., Leucine, denoted L in FIG. 4-1) while reference dye molecule 310B selectively binds to a second type of amino acid (e.g., Phenylalanine, Tyrosine, and Tryptophan, respectively denoted F, Y, and W in FIG. 4-1). Subsequent to sampling the sample molecule 309 (e.g., by delivering excitation light to the reaction chamber 308 and collecting signals emitted from the bound reference dye molecule 310A, 310B), cleavage of the sample molecule and/or the reference dye molecules 310A, 310B bound thereto may be performed with cleaving molecules 316. The sample molecules may be identified based on signals emitted by the reference molecules. The process of binding, receiving emission signals, and cleaving may be repeated multiple times for a single sample molecule and/or reaction chamber. In some embodiments, the calibration techniques described herein may be used to calibrate a device that is configured for use in protein and/or DNA/RNA sequencing applications.

During sequencing, emissions from molecules of interest may be obtained. Information derived from the emissions signals (e.g., signal intensity, fluorescence wavelength, fluorescence lifetime, etc.) may be used to identify the sample molecule. For example, FIG. 4-2 illustrates an example graph illustrating measured intensity and lifetime of emissions from sample molecules, according to some embodiments. FIG. 4-2 illustrates two clusters, 400A, 400B of emissions. The respective emissions clusters may be identified as belonging to a particular molecule or group of molecules based on the relative intensities and lifetimes of the emissions. For example, a first cluster 400A may be identified using the intensity and lifetime information as signals emitted from Leucine (L) or a reference dye attached thereto. A second cluster 400B may be identified based on intensity and lifetime information as signals emitted from one of Phenylalanine (F), Tyrosine (Y), or Tryptophan (W) or a reference dye attached thereto.

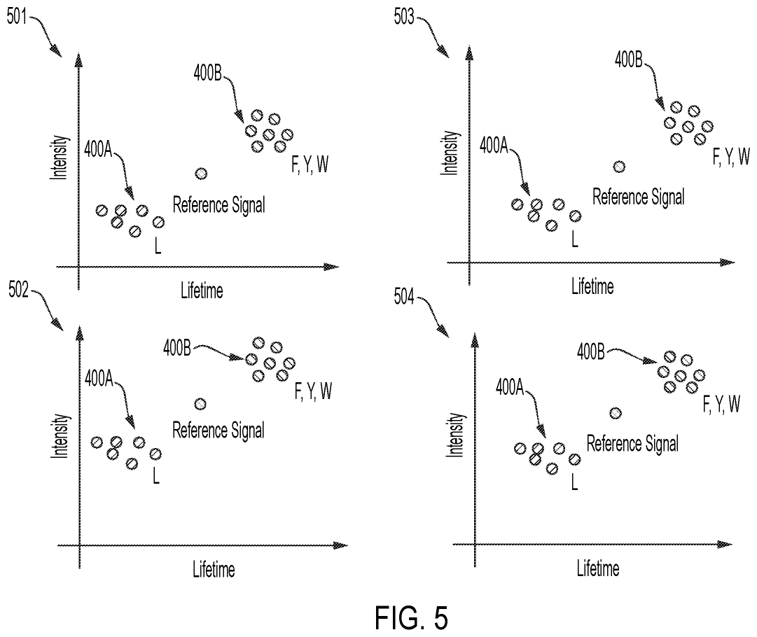

As described herein, the inventors have recognized that the reference dye emission information may be used to account for differences between reaction chambers which may otherwise lead to inaccuracies in identifying sample molecules during sequencing. For example, FIG. 5 illustrates example graphs 501-504 illustrating measured intensity and fluorescence lifetime of emissions from sample molecules in different reaction chambers, according to some embodiments. In particular, each of the example graphs shown in FIG. 5 illustrate clusters of signals obtained from a reference chamber as well as a signal from a reference dye. As described herein, a same type of reference dye molecule may be used in each reference chamber in the process 200. Thus, any difference in emissions characteristics (e.g., signal intensity, fluorescence wavelength, fluorescence lifetime, pulse duration, interpulse duration and/or combinations thereof) illustrated between graphs 501-503 are expected to be a result of differences between pixels of the integrated device (e.g., collection efficiency, excitation intensity, photodetector properties). The same variation between reference dye emissions can be expected when sequencing is performed. As such, an offset for each emissions characteristic of each reaction chamber may be determined using emissions information obtained from a reference dye molecule and applied to subsequent signal analysis during sequencing in order to better distinguish between multiple samples. Thus, the reference dye information may be used to localize clusters of signals obtained during sequencing.

FIG. 5 illustrates different offsets that may be realized when reference dye emission information is obtained. For example, the reference signals in graph 504 are shifted to the right (having a relatively longer lifetime) and upwards (relative more intense) relative to the reference signal in graph 501. Accordingly, clusters 400A, 400B in a reaction chamber or set of reaction chambers corresponding to graph 504 can be expected to have a shift in signal to the right and upwards relative to signals from clusters 400A, 400B in a reaction chamber or set of reaction chambers corresponding to graph 501. Therefore, the increase in intensity of signals from reaction chambers corresponding to graph 504 may be attributed, in some instances, to differences among the reaction chambers (e.g., fabrication or alignment differences) as opposed to differences among the fluorescent molecules emitting the signals. Accordingly, the calibration process described herein allows for accurately identifying samples despite differences among reaction chambers and minimizing inaccuracies in identifying samples due to differences among reaction chambers.

The calibration techniques described herein may be used in combination with any suitable sampling technique to be performed subsequent to calibration. For example, in some embodiments, the calibration techniques described herein may be used in combination with techniques for sample multiplexing as described in in U.S. Pat. Application No. 63/105,185, filed Oct. 23, 2020, titled “SYSTEMS AND METHODS FOR SAMPLE PROCESS SCALING” under Attorney Docket Number R0708.70112US00, which is incorporated by reference in its entirety.

In some embodiments, the emissions information may be used to identify a number of reference dye molecules in a reaction chamber. For example, if two or more emission signals are observed, it may be determined that multiple reference dye molecules have been loaded into a reaction chamber. In some embodiments, the measure of the number of sample molecules in a reaction chamber may be used to include or exclude certain reaction chambers from subsequent analysis. In some embodiments, bleaching information may be collected for the reference dye molecules (e.g., by recording a time it takes for the reference dye to bleach). In some embodiments, the bleaching information may be used to identify a number of reference dye molecules in a reaction chamber.

In some embodiments, the calibration techniques may be used in combination with techniques for sample identification using machine learning, for example, as described in in U.S. patent application Ser. No. 16/900,582, filed Jun. 12, 2020, titled “TECHNIQUES FOR PROTEIN IDENTIFICATION USING MACHINE LEARNING AND RELATED SYSTEMS AND METHODS” under Attorney Docket Number R0708.70063US01, which is incorporated by reference in its entirety.

### IV. Equivalents and Scope

Having thus described several aspects and embodiments of the technology of the present disclosure, it is to be appreciated that various alterations, modifications, and improvements will readily occur to those of ordinary skill in the art. Such alterations, modifications, and improvements are intended to be within the spirit and scope of the technology described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. The transitional phrases “consisting of” and “consisting essentially of” shall be closed or semi-closed transitional phrases, respectively.

What is claimed is:

1. A method for calibrating an integrated device, the method comprising:
exciting, with light from at least one excitation source, a reference dye molecule;
obtaining a signal emitted by the reference dye molecule, the signal containing information representative of at least one characteristic of the reference dye molecule; and
adjusting one or more subsequent measurements obtained from a sample based on the information obtained from the signal emitted by the reference dye molecule to obtain one or more adjusted measurements.

2. The method of claim 1, wherein the at least one characteristic of the reference dye molecule comprises a signal intensity, a fluorescence wavelength, a fluorescence lifetime, a pulse duration, and/or an interpulse duration of the reference dye molecule.

3. The method of claim 1, wherein the one or more adjusted measurements are representative of a signal intensity, a fluorescence wavelength, a fluorescence lifetime, a pulse duration and/or an interpulse duration of the sample.

4. The method of claim 1, further comprising identifying the sample based at least in part on the one or more adjusted measurements.

5. The method of claim 1, wherein the sample comprises a polypeptide and identifying the sample comprises identifying one or more amino acids of the sample based at least in part on the one or more adjusted measurements, and identifying the polypeptide based at least in part on the one or more identified amino acids.

6. The method of claim 1, wherein the sample comprises a strand of deoxyribonucleic acid (DNA) strand and identifying the sample comprises identifying one or more nucleotides of the DNA strand based at least in part on the one or more adjusted measurements.

7. The method of claim 1, wherein adjusting the one or more subsequent measurements comprises determining an offset to be added to the one or more subsequent measurements.

8. The method of claim 1, wherein the reference dye molecule comprises a fluorescent molecule immobilized in the chamber of the integrated device.

9. The method of claim 1, wherein:
the integrated device comprises a plurality of chambers;
the reference dye molecule comprises a plurality of reference dye molecules disposed in respective ones of the plurality of chambers; and
obtaining a signal emitted by the reference dye molecule comprises obtaining a plurality of signals emitted by the plurality of reference dye molecules;
the sample comprises a plurality of samples; and
adjusting the one or more subsequent measurements from the sample comprises adjusting one or more subsequent measurements obtained respectively from each of the plurality of samples based on information obtained from the plurality of signals emitted by the plurality of reference dye molecules.

10. The method of claim 9, wherein the plurality of reference dye molecules comprises a same molecule.

11. An integrated device comprising:
at least one chamber for receiving a reference dye molecule;
at least one photodetection region for receiving a signal emitted by the reference dye molecule when excited by light from at least one excitation source, the signal containing information representative of at least one characteristic of the reference dye molecule; and
at least one controller configured to obtain one or more adjusted measurements by controlling adjusting of one or more subsequent measurements obtained from a sample disposed in the at least one chamber based on the information obtained from the signal emitted by the reference dye molecule.

12. The integrated device of claim 11, wherein the at least one characteristic of the reference dye molecule comprises a signal intensity, a fluorescence wavelength, a fluorescence lifetime, a pulse duration, and/or an interpulse duration of the reference dye molecule.

13. The integrated device of claim 11, wherein the one or more adjusted measurements are representative of a signal intensity, a fluorescence wavelength, a fluorescence lifetime, a pulse duration, and/or an interpulse duration of the sample.

14. The integrated device of claim 11, wherein the at least one controller is further configured to identify the sample based at least in part on the one or more adjusted measurements.

15. The integrated device of claim 11, wherein the sample comprises a polypeptide and the at least one controller is configured to identify the sample at least in part by identifying one or more amino acids of the sample based at least in part on the one or more adjusted measurements, and identify the polypeptide based at least in part on the one or more identified amino acids.

16. The integrated device of claim 11, wherein the sample comprises a deoxyribonucleic acid (DNA) strand and the at least one controller is configured to identify the sample at least in part by identifying one or more nucleotides of the DNA strand based at least in part on the one or more adjusted measurements.

17. The integrated device of claim 11, wherein the at least one controller is configured to control the adjusting of the one or more subsequent measurements at least in part by determining an offset to be added to the one or more subsequent measurements.

18. The integrated device of claim 11, wherein:
the at least one chamber comprises a plurality of chambers;
the reference dye molecule comprises a plurality of reference dye molecules disposed in respective ones of the plurality of chambers;
the at least one photodetection region comprises a plurality of photodetection regions configured to receive signals from the plurality of reference dye molecules; and
the sample comprises a plurality of samples, wherein the at least one controller is configured to control the adjusting of the one or more subsequent measurements obtained respectively from each of the plurality of samples based on information obtained from the plurality of signals emitted by the plurality of reference dye molecules.

19. The integrated device of claim 18, wherein the plurality of reference dye molecules comprises a same molecule.

20. A method of manufacturing an integrated device, comprising:
providing a chamber on a substrate of the integrated device, the chamber being configured for receiving a reference dye molecule and positioned on the substrate such that the reference dye molecule receives light from at least one light source;
providing a photodetection region positioned adjacent to the chamber such that the photodetection region receives a signal emitted by the reference dye molecule when the light from the at least one light source is delivered to the reference dye molecule;
coupling at least one controller to the photodetection region so that the at least one controller receives the signal emitted by the reference dye molecule, the at least one controller being configured to obtain information representative of at least one characteristic of the reference dye molecule from the signal; and wherein the at least one controller is further configured to control adjusting one or more subsequent measurements obtained from a sample based on the information obtained from the signal emitted by the reference dye molecule to obtain one or more adjusted measurements.
